(19) United States
(12) Patent Application Publication
(10) Pub. No.: US 2005/0137603 A1
(43) Pub. Date: Jun. 23, 2005

(54) Acetabular cup positioning instrument

(76) Inventors: Kevin Belew, Hernando, MS (US); Amit Mistry, Collierville, TN (US); Jeff Shea, Memphis, TN (US); Justin Merritt Waugh, Memphis, TN (US)

Correspondence Address: CHIEF PATENT COUNSEL;SMITH & NEPHEW, INC., 1450 BROOKS ROAD, MEMPHIS, TN, 38116, US

(21) Appl. No.: 10/988,005

(22) Filed: Nov. 12, 2004

Related U.S. Application Data
(60) Provisional application No. 60/519,216, filed on Nov. 12, 2003.

(51) Int. Cl. A61B 17/58

(52) U.S. Cl. 606/91

(57) ABSTRACT

The present inventions relate to improved acetabular cup positioning instruments and improved methods of positioning an acetabular cup. One embodiment of the present inventions is an acetabular cup positioning instrument that utilizes suction to form an attachment with an acetabular cup and a stabilization component to stabilize the connection between the acetabular cup positioning instrument and the acetabular cup.

### RELATED APPLICATION DATA

This application claims the benefit of U.S. Ser. No. 60/519,216 filed Nov. 12, 2003 entitled “Acetabular Cup Positioning Instrument,” incorporated herein by this reference.

### FIELD OF INVENTION

The invention relates to apparatus for positioning a prosthetic acetabular cup within an acetabulum during hip replacement surgery.

### BACKGROUND

The hip is a ball-and-socket joint comprised of the head of the femur, the acetabulum of the pelvis, and ligaments of the hip joint. The head of the femur articulates or moves within the cup-like socket, the acetabulum, of the pelvic bone. The femoral head and acetabulum are typically covered by articular cartilage that allows smooth and painless motion of the hip joint. Hip replacement can benefit individuals suffering from a variety of hip problems including osteoarthritis, rheumatoid arthritis, traumatic arthritis, avascular necrosis, benign and malignant bone tumors, Paget's disease, and others.

Generally, during total hip replacement surgery, the hip joint is exposed and the head and neck of the femur are removed. The shaft of the femur is then prepared by reaming and/or broaching to accept a femoral implant typically consisting of a head, neck, and stem, which are usually made of plastic, metal or a combination of plastic and metal. The acetabulum is then reamed to accept a cup usually made of plastic or metal. Both of these implants may be fastened into the bone with or without special cement. The cemented procedure utilizes a doughy substance mixed at the time of surgery that is introduced between the artificial component and the bone. Cementless hip replacement procedures attach the acetabular cup without using cement. For example, the procedure may use an artificial joint covered with a material that allows bone tissue to grow into the metal forming a tight bond of scar tissue which anchors the metal to the bone.

Acetabular cup positioners are used during hip arthroplasty to position the prosthetic acetabular cup within the prepared acetabulum of the patient. Placement of the acetabular cup during surgery can be a difficult task. The surgeon must adequately fix the cup in the proper alignment with limited surgical exposure and few clear reference points. Surgeons typically attach an acetabular cup positioner to the acetabular cup, position the acetabular cup within the prepared acetabulum using that instrument, and then detach the instrument from the cup. Occasionally, an acetabular cup will fall off of the positioning instrument during insertion, requiring the surgeon to reattach the cup to the positioning instrument.

Acetabular cup positioning instruments provide a variety of ways to accomplish attaching the positioning instrument to the cup. For example, some designs hold the acetabular cup to the acetabular positioner using an interference fit between two pins located on the positioner head and two corresponding pin holes located on the face of the acetabular cup component. Designs of this type are useful but may lead to difficulties when reattachment is required when an acetabular cup falls off. Other designs have used an interference fit between a silicone nipple, located on the distal tip of the positioner, and the articulating surface of the acetabular cup component. These designs often do not hold the acetabular cup component securely and do not offer adequate control of the acetabular cup component.

Another common problem associated with surgical use of acetabular cup positioners is that removal of the positioner from the cup occasionally disrupts the position of the acetabular cup component, requiring the surgeon to reposition the cup component. For example, for the two pin interference designs described above, the acetabular cup component may be pressed onto the positioner head and is ordinarily pushed off with an extractor rod. The pressure applied by the extractor rod has the potential to disrupt the location of the acetabular cup. Other types of positioners are removed or detached by applying various types of physical force to separate the positioner from the cup. Many acetabular cup positioners suffer significant disadvantages because of the difficultly and disruptiveness necessary to attach, detach, and reattach these positioners to the acetabular cup components.

The use of a vacuum or suction as part of a positioning or inserting instrument provides some benefits including the ability to detach the positioner from the cup with reduced disruption. See U.S. Pat. No. 3,859,992 to Amstutz and German patent DE 101 28 234 to Anmelder. However, the use of vacuum or suction to attach an acetabular cup positioning instrument to an acetabular cup does not provide adequate stability, rotational control, or leverage to allow a surgeon to adequately manipulate the cup into its proper or otherwise desired position. Moreover, attachment by vacuum or suction alone does not provide the secure and stable connection necessary to push, hammer, or press fit an acetabular cup or insert into place using an acetabular cup positioning instrument.

### SUMMARY

The present invention relates to improved acetabular cup positioning instruments and improved methods of positioning an acetabular cup. One embodiment of the present inventions is an acetabular cup positioning instrument that utilizes suction to form an attachment with an acetabular cup and a stabilization component to stabilize the connection between the acetabular cup positioning instrument and the acetabular cup. The suction may be provided by a suction cup that interfaces with the articulating surface of the acetabular cup. A surgeon using the acetabular cup positioning instrument can control the suction to attach and detach the acetabular cup positioning instrument to the acetabular cup without causing significant disturbance to the acetabular cup. The stabilization component may provide stability, rotational control, leverage, and/or additional connective strength and allow the transmission of force from the instrument to the cup. The combination of a suction component and a stabilization component on the acetabular cup positioning instrument allows a surgeon using the instrument to properly position an acetabular cup using the instrument and then detach the instrument from the acetabular cup without significantly disturbing the position, angle, or direction of the acetabular cup. This is particularly advantageous in applications involving wet cement in which the acetabular cup is sensitive to disruption.

### BRIEF DESCRIPTION OF THE DRAWINGS

FIG. 1 illustrates an acetabular cup positioning instrument in accordance with one embodiment of the present invention and an exemplary acetabular cup component set in the acetabulum of a model pelvic bone.

FIG. 2 illustrates an acetabular cup positioning instrument in accordance with one embodiment of the present invention.

FIG. 3 illustrates an acetabular cup positioning instrument in accordance with one embodiment of the present invention.

FIG. 4 illustrates the handle of an acetabular cup positioning instrument in accordance with one embodiment of the present invention.

FIG. 5 illustrates the handle of an acetabular cup positioning instrument in accordance with one embodiment of the present invention.

FIG. 6 illustrates the handle of an acetabular cup positioning instrument in accordance with one embodiment of the present invention.

FIG. 7 illustrates a method of attaching an acetabular cup to an acetabular cup positioning instrument in accordance with one embodiment of the present invention.

FIG. 8 illustrates a method of inserting an acetabular cup into an acetabulum using an acetabular cup positioning instrument in accordance with one embodiment of the present invention.

FIGS. 9a and b illustrate one end of an acetabular cup positioning instrument in accordance with one embodiment of the present invention.

FIG. 10 illustrates a stabilization component on one end of an acetabular cup positioning instrument in accordance with one embodiment of the present invention.

FIG. 11 illustrates a method of separating the components of an acetabular positioning instrument for cleaning or sterilization according to one embodiment of the present invention.

FIG. 12 illustrates a positioning instrument used with a metal acetabular cup according to one embodiment of the present invention.

FIG. 13 illustrates a positioning instrument used with a metal acetabular cup according to one embodiment of the present invention.

### DETAILED DESCRIPTION

One embodiment of the present invention includes an acetabular cup positioning instrument with a shaft, a suction component, and a stabilization component. The suction component may be found at one end of the shaft and allows attachment of the acetabular cup positioning instrument to an acetabular cup. The suction component may allow a releasable connection to the acetabular cup. It also allows the transmission of force from the positioner to the cup. The stabilization component provides stability, rotational control, and leverage control of the acetabular cup by interfacing with the acetabular cup. The stabilization component may accomplish the stabilization function in a variety of ways. In one embodiment, the stabilization component is designed to interface with the end of the acetabular cup limiting the directions the cup can rotate, twist, or bend. In another embodiment, the stabilization component provides stability as a spring-loaded plate that limits, but does not prevent, movement of the attached acetabular cup.

Another embodiment of the present invention is a method of positioning an acetabular cup within an acetabulum during hip replacement surgery. This method involves using an acetabular cup positioning instrument having a suction component and a stabilization component to position and insert the acetabular cup. The acetabular cup is attached to the acetabular cup positioning instrument using suction provided by the suction component. Stabilization is created by interfacing the stabilization component with the acetabular cup. Once the cup is attached and stabilized, the acetabular cup positioning instrument is used to properly position the acetabular cup. Finally, once the acetabular cup is properly inserted, the acetabular cup is detached from the acetabular cup positioning instrument by releasing the suction provided by the suction component. This method of detachment will typically provide significantly less disturbance to the position of the acetabular cup than other, more-mechanical methods of detachment.

The suction component of the present invention may be in a variety of forms, sizes, shapes, and provide suction in a variety of ways. For example, in some embodiments, the suction component is a suction cup. In some embodiments, the suction component induces at least a partial vacuum. In still other embodiments, the suction component functions as a dent puller, where plastic material of the suction component has a similar shape to a portion of the articulating surface of an acetabular cup. In this embodiment, the plastic of the suction component is placed against the articulating surface of the acetabular cup and suction is created by mechanically pulling a portion of the plastic away from the articulating surface. This dent puller type suction component may provide stronger suction and, therefore, may be particularly useful for use with heavier metal acetabular cup components and in metal to metal hip replacement surgery discussed below.

The stabilization components of different embodiments of the invention may provide one or more specific stabilization functions or a combination of stabilization functions. Stabilization functions include but are not limited to providing rotational control, providing leverage control, and providing transmission of force. Rotational control allows the surgeon to control the rotation and rotational position of the acetabular cup using the acetabular cup positioning instrument. Typically, the surgeon can rotate the acetabular cup by rotating the positioning instrument. This function may be accomplished by providing an interface between the positioning instrument and the cup that rotationally links these components. For example, a protrusion in the positioning instrument may fit into a recess in the acetabular cup such that when the instrument is rotated along its center axis, the cup rotates along with it.

Leverage control, as the name suggests, allows the surgeon to manipulate the position of the acetabular cup using leverage. Leverage control is created by having the positioning instrument securely interface with the acetabular cup such that as the positioning instrument is adjusted, moved, or titled in different directions the acetabular cup moves with the instrument. This gives the surgeon sufficient control over the acetabular cup to insert it into a desired position.

The stabilization component of the positioning instrument may also provide stability by allowing the surgeon to transmit force to the acetabular cup using the positioning instrument. This is accomplished by having the positioning instrument interface with the acetabular cup in a way that allows force to be transmitted without detaching the acetabular cup or having it twist, bend, tilt, or otherwise move into an undesired position. For example, a surgeon may push or hammer on the shaft or handle of a positioning instrument to press the acetabular cup into the prepared acetabulum. As the surgeon pushes, the interface transfers the force to the acetabular cup and prevents it from twisting, bending, tilting, or otherwise moving into an undesired position.

In some embodiments, the leverage and force transmission capability of the stabilization component is provided in a plate that interfaces with a face of an acetabular cup. The face or rim of the acetabular cup is a portion or edge that is typically flat and surrounds the perimeter of the articulating surface of the acetabular cup. Examples of face components of acetabular cups are described in the figures below. The term “face” as used herein with respect to acetabular cup components should not be limited to a particular shape, orientation, size, or angle. The term is generally used to describe a portion of the acetabular cup other than the articulating surface that the positioning instrument may interface with in some way.

The acetabular cup positioning instruments and methods of the present invention are useful with a variety of types of acetabular cups and applications for those cups. The instruments may be used with acetabular cups made from many different types of materials, including plastics, metals, and combinations of materials. The inventions are not limited to use with acetabular cups made of any particular material or combination of materials. Accordingly, the instruments may be used in applications in which the acetabular cup is made entirely of plastic.

The instruments and methods of the present invention may also be used in metal to metal hip replacement surgeries. Recent technological advances are allowing the reintroduction of metal to metal hip replacement surgery. These procedures are currently growing in popularity in Europe and are likely to be reintroduced in the United States. Metal to metal hip replacement devices and techniques typically involve hip replacement systems where both the replacement femoral head and the replacement acetabular cup are made of metal, although not necessarily the same type of metal. The metal of the femoral head interacts directly with the metal of the acetabular cup.

The instruments and methods of the present invention may also be useful to insert a non-metal or metal insert component into a separate acetabular cup component.

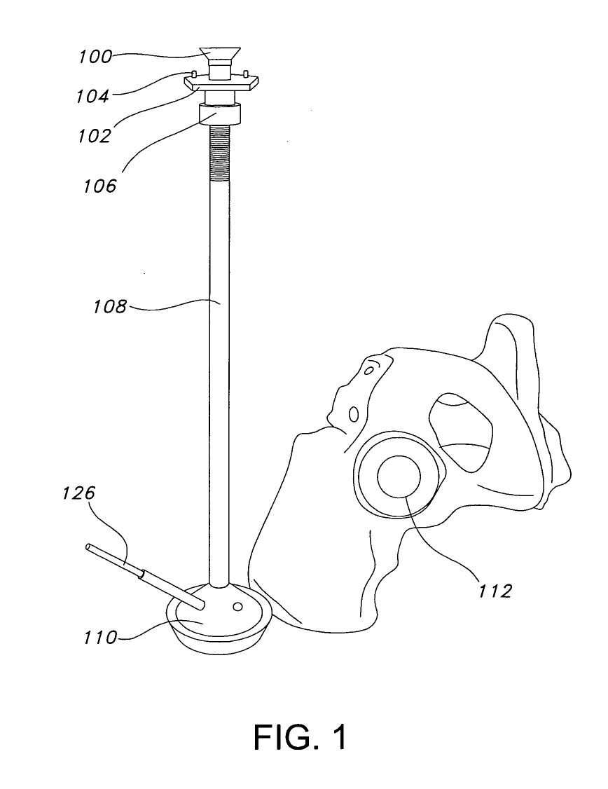

FIG. 1 illustrates an acetabular cup positioning instrument in accordance with one embodiment of the present invention and an exemplary acetabular cup component 112 set in the acetabulum of a model pelvic bone. In this embodiment, the acetabular cup positioning instrument includes a suction cup 100 as the suction component and an alignment guide 102 as the stabilization component. This embodiment also includes a shaft 108, a handle 100, an alignment rod 126, and an alignment guide securing component such as a threaded collar 106. The alignment guide 102 of the acetabular cup positioning instrument may also include one or more protrusions or pins 104 to provide rotational control.

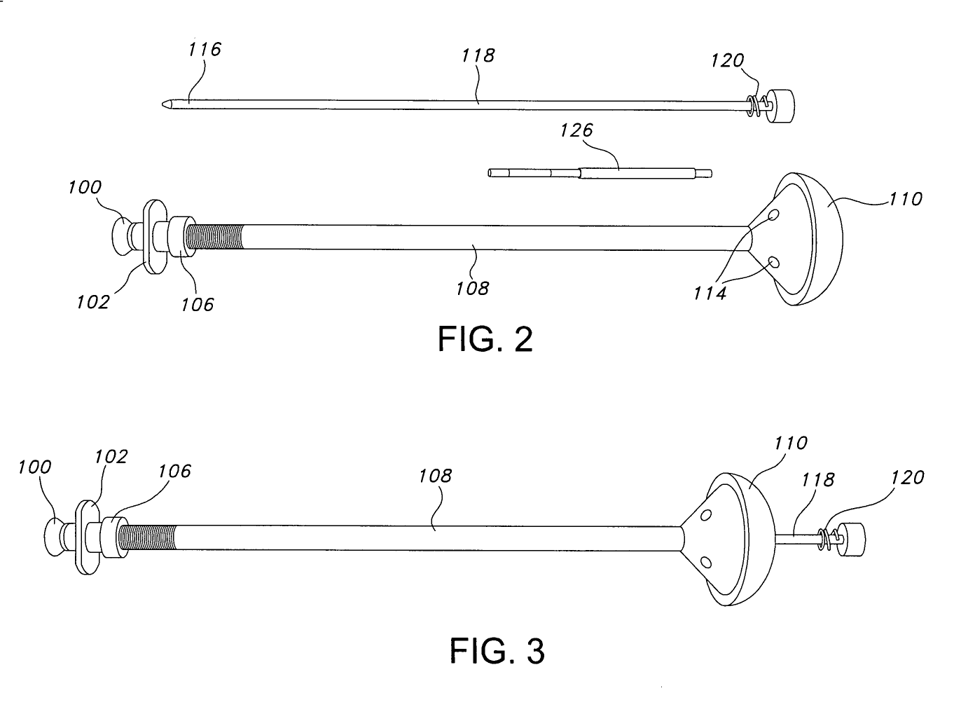

FIGS. 2 and 3 illustrate that, in one embodiment, the shaft 108 may be an outer sleeve designed to allow an inner sleeve 118 to pass through it. This inner sleeve 118 may be used to control the suction of the suction cup 100 or other suction component. In this embodiment, the inner sleeve 118 includes an o-ring or gasket 116 for providing an air tight seal within the outer sleeve of shaft 108. The inner sleeve 118 may also include a spring 120 at the opposite end of the inner shaft 118. In this embodiment, the spring 120 is used to control the position of the inner shaft 118 within the outer sleeve of the shaft 108, and thereby to control the suction of the suction cup 100 or other suction component. Once suction is created in the suction cup 100, the spring 120 prevents the inner shaft from shifting toward the suction cup end of the outer sleeve of the shaft 108, preserving the suction. However, the surgeon can press the end of the inner shaft 118 compressing the spring 120, pushing the inner shaft 118 toward the suction cup 100, moving air into the suction cup, and breaking the suction cup 100 seal. Accordingly, in this embodiment, the inner shaft 118 is used to control the suction and detach acetabular cup positioning instrument from the acetabular cup component.

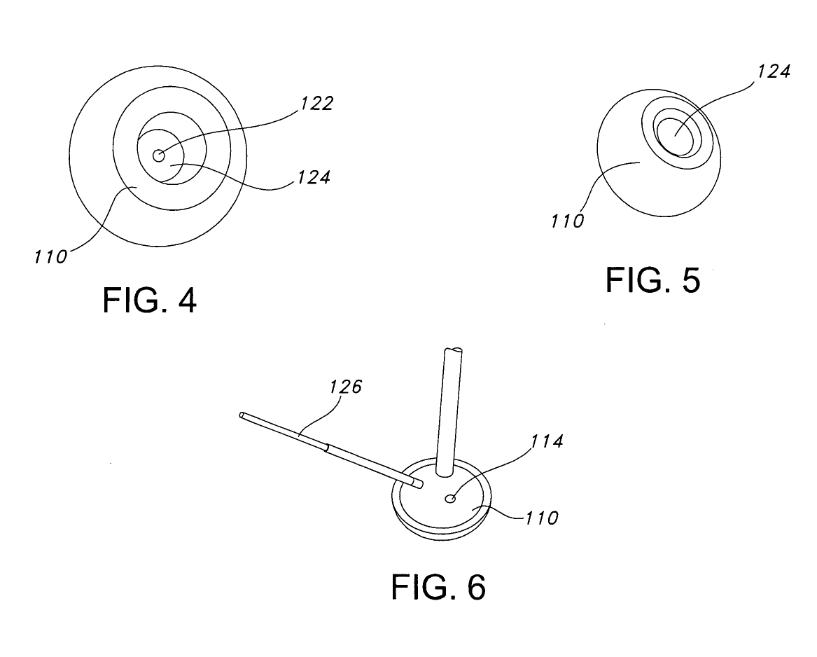

FIGS. 4 and 5 illustrate a handle 110 for use with the embodiment described above. The inner shaft 118 is inserted into hole 122 and spring 120 fits into recess 124. The inner shaft 118 and spring 120 may fit into the outer sleeve of the shaft 108 such that they do not interfere with the operation of the positioning instrument, but are accessible when detachment is desired. Accordingly, the inner shaft 118 may be recessed within the handle 110.

FIG. 6 illustrates the handle of an acetabular cup positioning instrument having alignment rod holes 114 for insertion of alignment rods. Alignment rod 126 may be inserted into one of alignment rod holes 114 to provide additional stability, alignment or control.

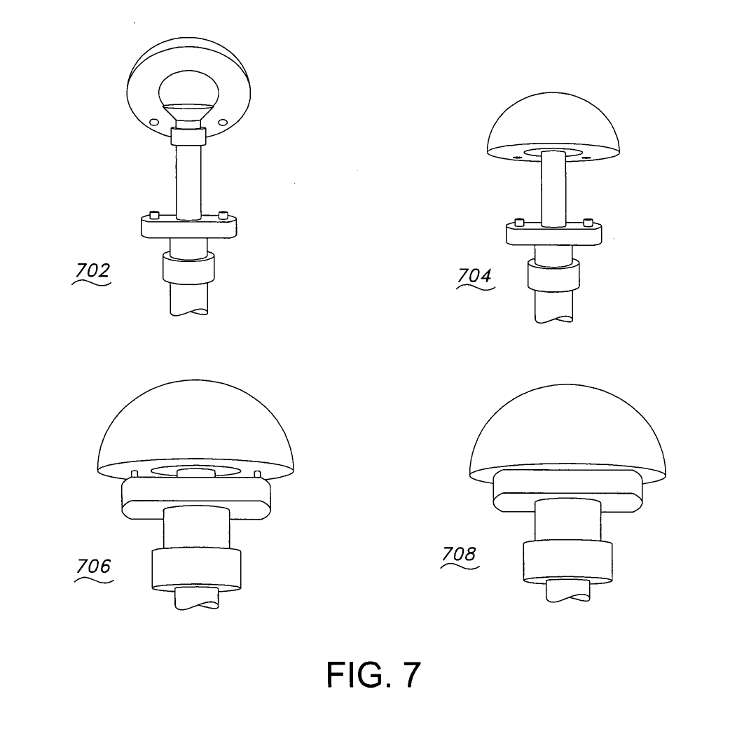

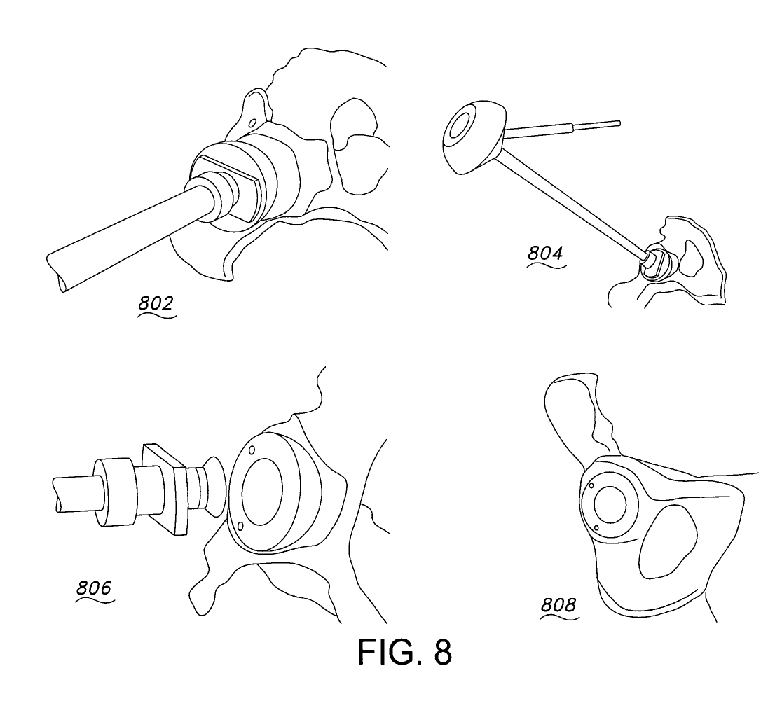

FIG. 7 illustrates a method of attaching an acetabular cup to an acetabular cup positioning instrument in accordance with one embodiment of the present invention. After checking to make sure that the alignment guide and threaded collar are all the way down in the load position, the first step 702 consists of aligning the holes or recesses in the acetabular cup with the protrusions or pins of the alignment guide of the acetabular cup positioning instrument. Step two 704 is pressing down on the acetabular cup to secure it to the section component. Step three 706 is to begin tightening the threaded collar. As the alignment component approaches that acetabular cup during tightening, ensure that the protrusions or pins on the alignment guide go into the holes of the acetabular cup, as shown in step four 708. Continue to tighten the threaded collar until tight and secure. The cup is now secure, loaded, and stabilized.

Once the cup is secured, an alignment rod 126 can be screwed in or otherwise attached to the handle into either a left or right hole 114 depending on which hip the cup is being implanted in.

FIG. 8 illustrates a method of inserting an acetabular cup into an acetabulum using an acetabular cup positioning instrument in accordance with one embodiment of the present invention. In the first step 802 the acetabular cup is positioned into the desired location at the desired angle. After the cup is properly placed, in step two 804 the cup is released by releasing the suction of the suction component. In this embodiment, the suction is released by pressing the inner shaft toward the suction cup. In the third step 806 the instrument is detached or removed from the acetabular cup. Finally, step four 808 shows the cup successfully implanted.

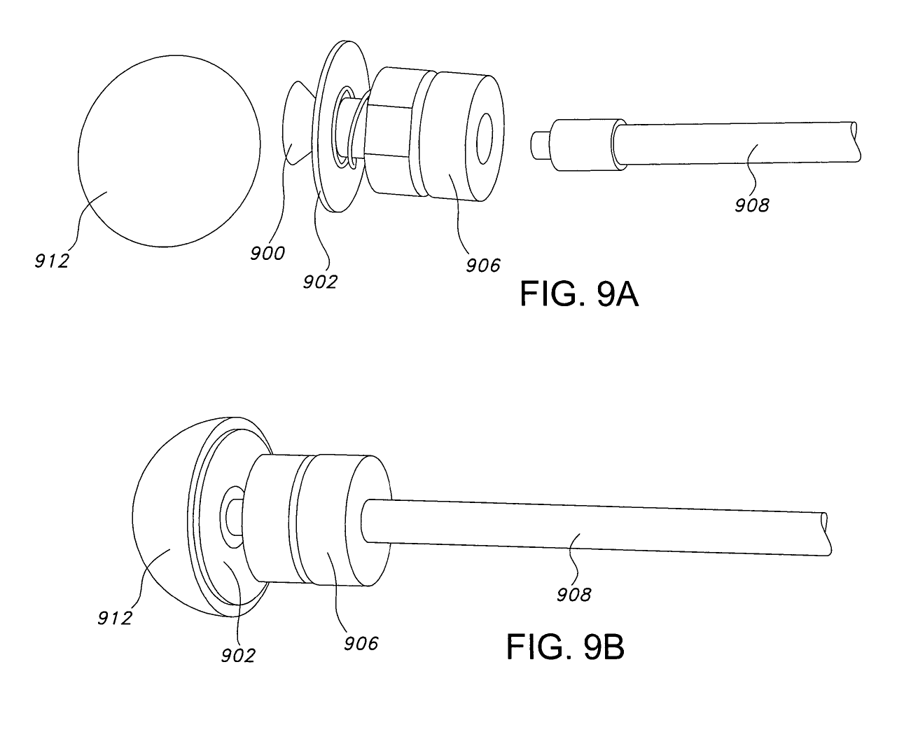

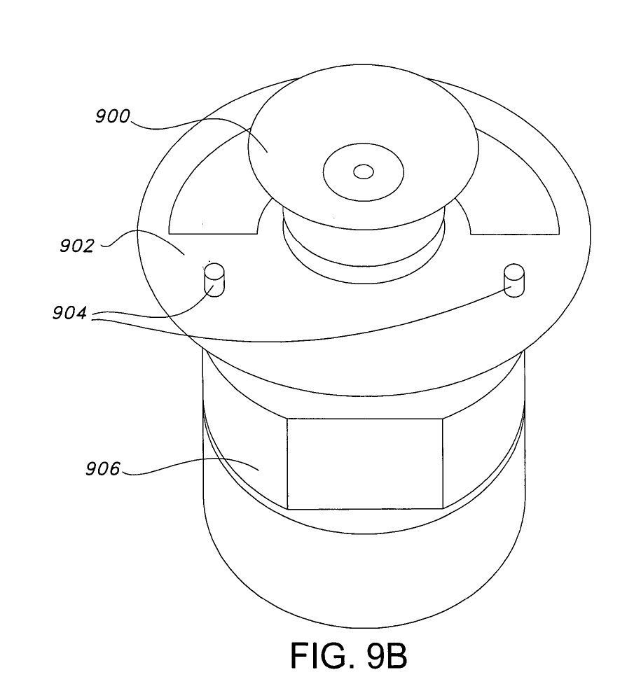

FIGS. 9a and b illustrate one end of an acetabular cup positioning instrument in accordance with one embodiment of the present invention. In the device shown, the suction creates the connection between the positioner 908 and the acetabular cup component 912 and may be controlled by the surgeon by adjusting the positioner device. In one embodiment, the positioner head has two settings to control the suction, “Lock” and “Release” 906. In the “Lock” setting, the positioner head provides an air-tight connection between the suction cup and the articulating surface. In the “Release” setting, the positioner head allows air into the suction cup thus eliminating the air-tight suction.

In FIGS. 9a and 9b the stabilization component 902 comprises a plate which stabilizes the connection between the acetabular cup positioning instrument 908. The stabilization plate 902 interfaces with a face of the acetabular cup. In this embodiment the plate is spring-loaded to provide stability with a small degree of flexibility. Alternatively, the plate may be held securely rather than spring-loaded providing more rigid stabilization.

In other embodiments, the stabilization component 902 interfaces with the articulating surface of the acetabular cup rather than with its face. The stabilization component 902 may have beveled edges to facilitate a stable interface with the articulating surface. Other shapes, sizes, and connections are envisioned for the stabilization component.

FIG. 10 illustrates stabilization components in the form of a plate 902 near one end of an acetabular cup positioning instrument. The stabilization component 902 has two protrusions or pins 904 projecting from its surface. These protrusions or pins may interface with corresponding holes in an acetabular cup to provide rotational control.

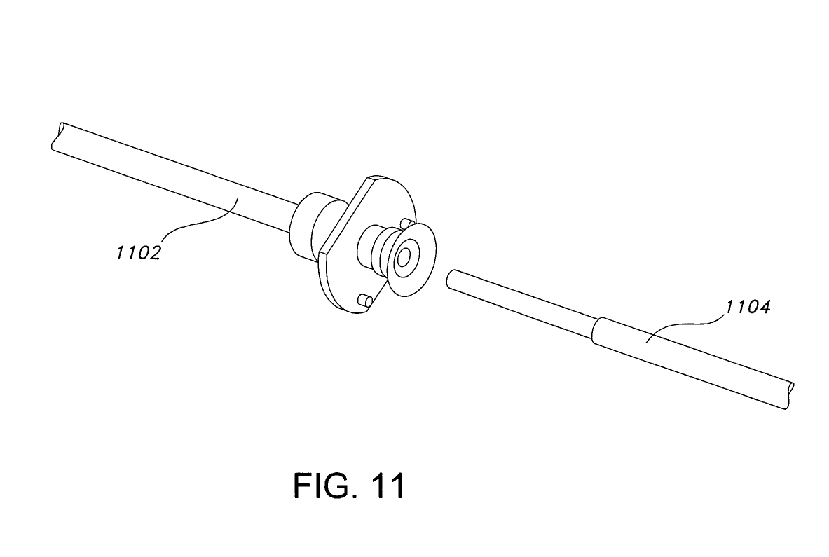

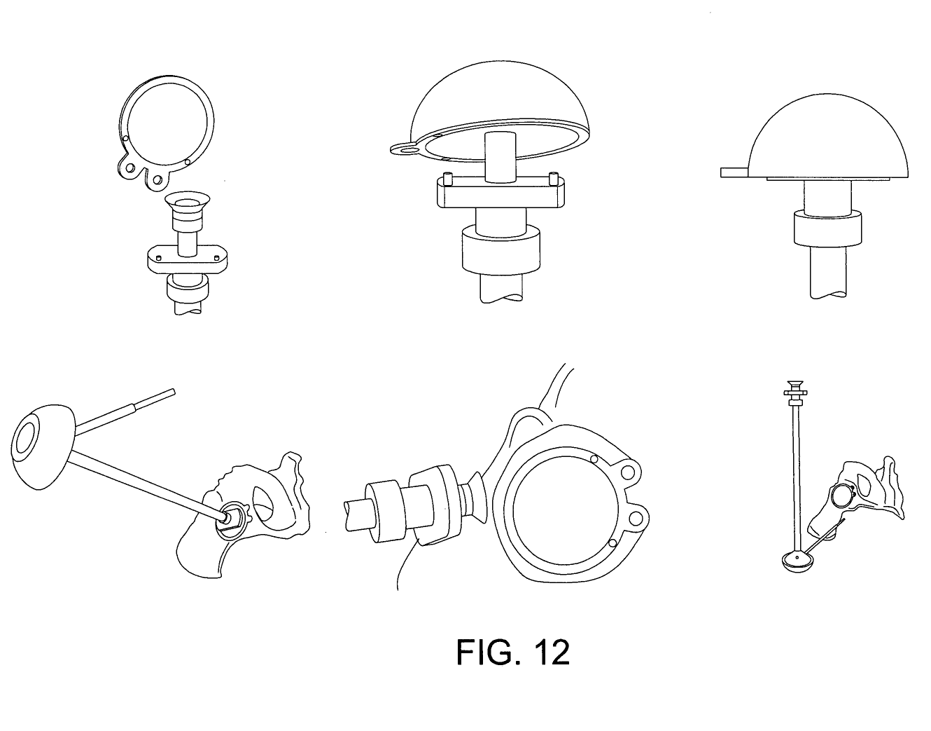

FIG. 11 illustrates a method of separating the components of an acetabular positioning instrument 1104 for cleaning or sterilization according to one embodiment of the present invention. One end of alignment rod 1104 may be inserted into the suction cup end of the acetabular cup positioning instrument 1102 to push out the inner shaft, not shown. In this manner, the inner shaft can be removed from the acetabular cup positioning instrument 1102 for cleaning. Designing orthopedic instruments that allow easy sterilization often presents significant challenges. The ability to easily separate components from one another after use provides many benefits. For example, the improved ability to thoroughly sterilize components for future use may extend the useful life of a given product.

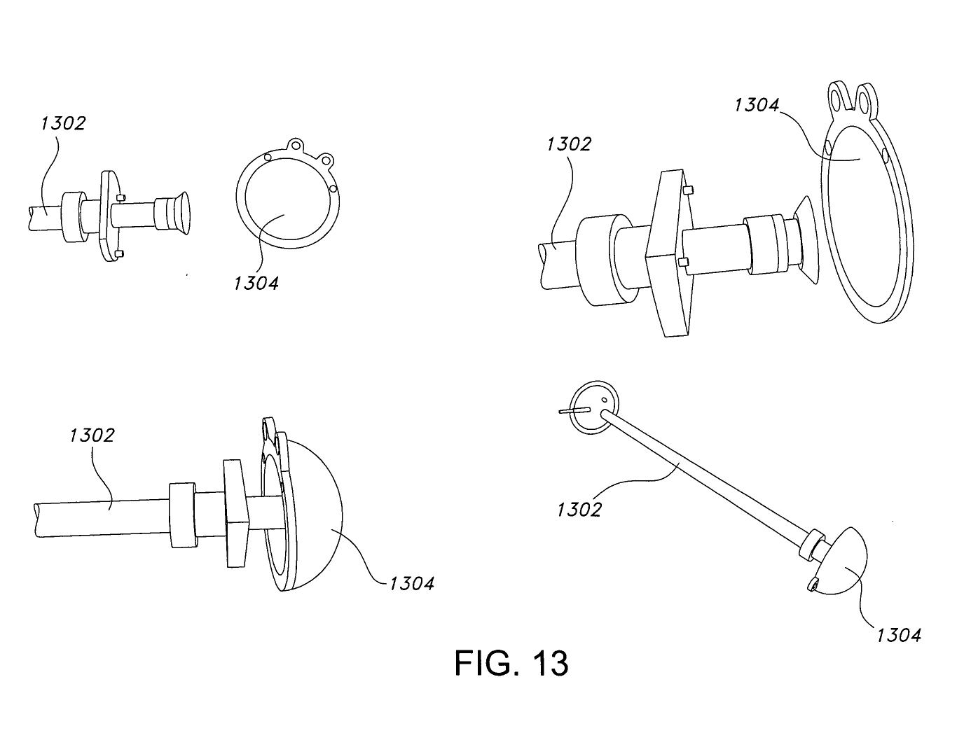

FIGS. 12 and 13 illustrate the use of an acetabular cup positioning instrument with a metal acetabular cup component in accordance with one embodiment of the present invention. As shown in FIG. 13, the acetabular cup positioning instrument 1302 attaches to and provides stability to the metal acetabular cup 1304.

Another aspect of the present invention is a method of using an acetabular cup positioning instrument during hip replacement surgery. This method involves using an acetabular cup positioning instrument having a suction component and a stabilization component similar to those in the instruments described above. The steps include attaching the acetabular cup to the acetabular cup positioning instrument using suction provided by the suction component, stabilizing the acetabular cup by interfacing the stabilization component with the acetabular cup, positioning the acetabular cup using the acetabular cup positioning instrument; and detaching the acetabular cup from the acetabular cup positioning instrument by releasing the suction provided by the suction component.

In certain embodiments, the method may also include stabilizing the acetabular cup by fitting a protrusion or pin of the stabilization component into a hole in the acetabular cup. In other embodiments, the method may include stabilizing the acetabular cup by tightening a threaded collar.

The structures and processes described above illustrate a preferred embodiment of inventive concepts included in the present invention. Other systems and processes are possible. While the invention has been described in detail with particular reference to this particular embodiment, variations and modifications can be effected within the spirit and scope of the invention as described in this document.

1. An acetabular cup positioning instrument comprising:
a shaft;
a suction component at one end of the shaft for attaching the acetabular cup positioning instrument to an acetabular cup, wherein the suction component allows a releasable connection to the acetabular cup; and
a stabilization component for providing an interface between the shaft and the acetabular cup, whereby the shaft can be used to manipulate orientation of the cup.

2. The instrument of claim 1, wherein the suction component is a suction cup that engages an articulating surface of the acetabular cup.

3. The instrument of claim 1, wherein the suction component induces at least a partial vacuum.

4. The instrument of claim 1, wherein the suction component is controlled by a lock/release component, wherein the lock/release component is adjustable to either prevent air from entering or allow air to enter the suction component.

5. The instrument of claim 4 further comprising a rubber gasket for allowing air to enter the suction component.

6. The instrument of claim 1, wherein the stabilization component includes a protrusion for interfacing with a recess in the acetabular cup.

7. The instrument of claim 1, wherein the stabilization component includes two protrusions for interfacing with two recesses in the acetabular cup and for providing rotational control of the acetabular cup.

8. The instrument of claim 1, wherein the stabilization component is secured to the acetabular cup using an alignment guide securing component.

9. The instrument of claim 1, wherein the alignment guide securing component is a threaded collar.

10. The instrument of claim 1, wherein the shaft comprises an outer sleeve and wherein an inner shaft is insertable into the outer sleeve for controlling the suction provided by the suction component.

11. The instrument of claim 10, wherein the inner shaft includes a gasket.

12. The instrument of claim 10, wherein the inner shaft includes a spring.

13. The instrument of claim 1, wherein the handle includes alignment rod holes.

14. The instrument of claim 1, wherein the stabilization component comprises a plate which stabilizes the connection between the acetabular cup positioning instrument, wherein the plate interfaces with a face of the acetabular cup.

15. The instrument of claim 14, wherein the plate is a spring-loaded plate.

16. The instrument of claim 1, wherein the stabilization component comprises a plate which stabilizes the connection between the acetabular cup positioning instrument, wherein the plate interfaces with an articulating surface of the acetabular cup.

17. The instrument of claim 15, wherein the plate comprises beveled surfaces for interfacing with the articulating surface of the acetabular cup.

18. The instrument of claim 1 wherein the stabilization component provides rotation control of the cup via the shaft.

19. The instrument of claim 1 wherein the stabilization component provides leverage control of the cup via the shaft.

20. The instrument of claim 1 wherein the stabilization component assists in transmission of force from the shaft to the cup.

21. A method of positioning an acetabular cup within an acetabulum during hip replacement surgery comprising:
providing an acetabular cup positioning instrument having a suction component and a stabilization component;
attaching the acetabular cup to the acetabular cup positioning instrument using suction provided by the suction component;
stabilizing the acetabular cup by interfacing the stabilization component with the acetabular cup;
positioning the acetabular cup using the acetabular cup positioning instrument; and
detaching the acetabular cup from the acetabular cup positioning instrument by releasing the suction provided by the suction component.

22. The method of claim 21, wherein the step of stabilizing the acetabular cup further comprises fitting a protrusion of the stabilization component into a recess in the acetabular cup.

23. The method of claim 21, wherein the step of stabilizing the acetabular cup further comprises tightening a threaded collar to provide additional stability.

24. The method of claim 21 further comprising installing a femoral component and coupling the femoral component to the cup.
